(19) United States
(12) Patent Application Publication
(10) Pub. No.: US 2010/0300454 A1
(43) Pub. Date: Dec. 2, 2010

(54) Limb positioning device for use in diagnostic imaging

(76) Inventor: Kelly D. Brewer, Carlsbad, NM (US)

Correspondence Address: Alexander B. Ching, Suite 212, 1601 North Turner, Hobbs, NM, 88240, US

(21) Appl. No.: 12/455,233

(22) Filed: May 28, 2009

(51) Int. Cl. A61B 6/04

(52) U.S. Cl. 128/845

(57) ABSTRACT

A device for positioning a limb for diagnostic imaging comprises a first bag configured to be placed against one side of the limb and a second bag configured to be placed against the second side of a limb. A web section connects the first bag and the second bag. The web section is placed underneath the limb. A plurality of straps with a portion of the plurality of straps connected to the first bag and the remainder of the plurality of straps connected to the second bag are configured to secure the device to the limb.

### FIELD OF THE INVENTION

The present invention generally relates to the field of medical devices and more specifically to a limb positioning device for use in diagnostic imaging.

### BACKGROUND OF THE INVENTION

Great advances in medical imaging have been made over the years. Where there were once only x-rays, we now have CT scans and MRIs. These advances have help doctors provide more sophisticated diagnosis and have enhanced the treatment of patients in many areas of medicine. However, no matter how sophisticated the imaging method, the end results are dependent on, among other factors, how stable the patient holds the body part that is being imaged. This is true of x-rays, MRI and CT scans; if the patient moves the quality of the image will suffer. While imaging methods have become increasing sophisticated, imaging technicians rely on low tech methods to help position and/or immobilize a patient's limb for imaging. For example, sand bags and foam wedges are typically used to help position and immobilize limbs for MRI imagining. Drawbacks to this approach includes the discomfort of placing a sandbag on top of a limb to help position it as well as the inconvenience and time spend placing various sandbags for optimal effect.

Accordingly, it is desirable to provide a limb positioning device for use in diagnostic imaging. Furthermore, other desirable features and characteristics of the present invention will become apparent from the subsequent detailed description of the invention and the appended claims, taken in conjunction with the accompanying drawings and this background of the invention.

### BRIEF SUMMARY OF THE INVENTION

In one embodiment of the present invention a device for positioning a limb for diagnostic imaging comprises a first bag configured to be placed against one side of the limb and a second bag configured to be placed against the second side of a limb. A web section connects the first bag and the second bag. The web section is placed underneath the limb. A plurality of straps with a portion of the plurality of straps connected to the first bag and the remainder of the plurality of straps connected to the second bag are configured to secure the device to the limb.

### BRIEF DESCRIPTION OF THE DRAWINGS

The present invention will hereinafter be described in conjunction with the following drawing figures, wherein like numerals denote like elements, and:

FIG. 1 illustrates a device for positioning a limb in accordance with the teachings of the present invention;

FIG. 2 is a cross section of a device for positioning a limb in accordance with the teachings of the present invention;

FIG. 3 illustrates a device for positioning a limb in use with an imaging system in accordance with the teachings of the present invention; and

FIG. 4 is a flowchart of an exemplary method for using the device in accordance with the teachings of the present invention.

### DETAILED DESCRIPTION OF THE INVENTION

The following detailed description of the invention is merely exemplary in nature and is not intended to limit the invention or the application and uses of the invention. Furthermore, there is no intention to be bound by any theory presented in the preceding background of the invention or the following detailed description of the invention.

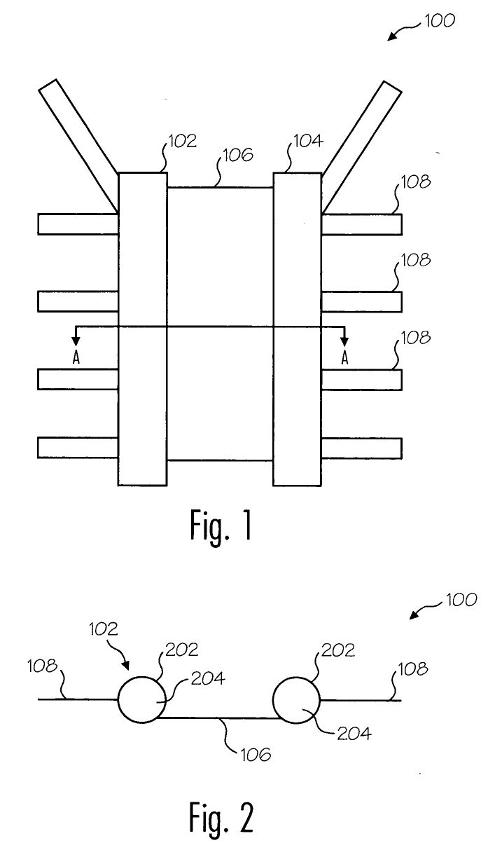

FIG. 1 illustrates an exemplary device 100 in accordance with the teachings of the present invention. Device 100 is designed to position a limb, such as the leg or arm of a patient, when the patient is undergoing medical diagnostic imaging, such as an MRI or a CT scan. Device 100 can also be utilized to position a portion of a limb. In one exemplary embodiment, device 100 positions and immobilizes a limb for a patient undergoing diagnostic imaging. As illustrated in FIG. 1, device 100 comprises a first bag 102 and a second bag 104 connected by a webbing section 106. A plurality of straps 108 are connected to each of the first and second bags 102 and 104.

First and second bag 102 and 104 provide support to position and hold the limb of a patient in a set position for use in a medical imagining device. In one embodiment, first and second bag 102 and 104 are approximately cylindrical or rectangular in shape, although other shapes that fit along a limb to hold the limb in place can be used. As seen in the cross section of device 100 in FIG. 2, first bag 102 and second bag 104 comprises a cover portion 202 and a filler material 204.

Cover portion 202 can be any material that is strong enough to hold in filler section 204. Cover portion 202 preferably is made of a durable, easy to clean material such as vinyl. Easy to clean materials are advantageous in a medical setting as the device would need to be cleaned and sanitized.

Filler material 204 can be any material or medium having sufficient weight to, when placed adjacent to a limb, immobilize a limb in a fixed position. Filler material 204 is also preferable a malleable material that can be shaped to conform to the shape of a limb. In one example, the filler material is sand. In another exemplary embodiment, the sand is a non-metallic sand that has no metallic material such that device 100 can be used with an MRI. In yet another embodiment, filler material 204 is a PVC sand, which is a sand-like material made from plastic. Filler material 204 can also be a gel or similar material having a sufficient density and weight to immobilize a limb or portion thereof.

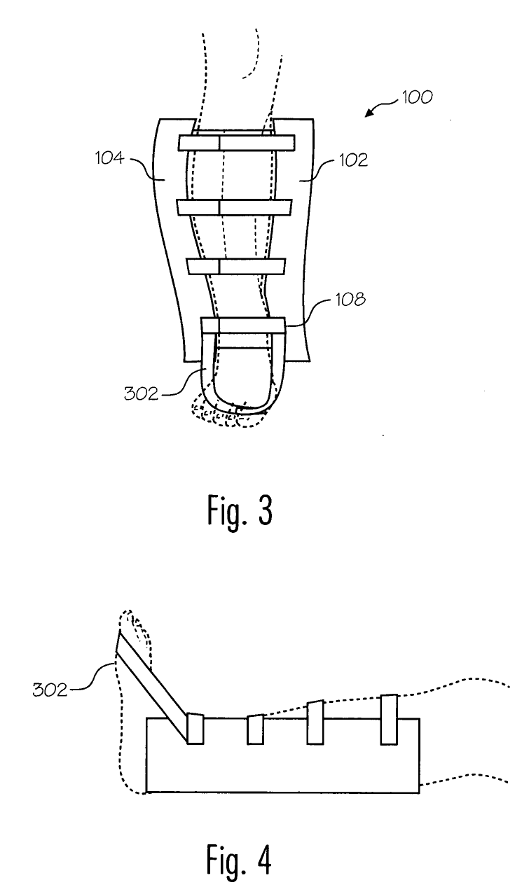

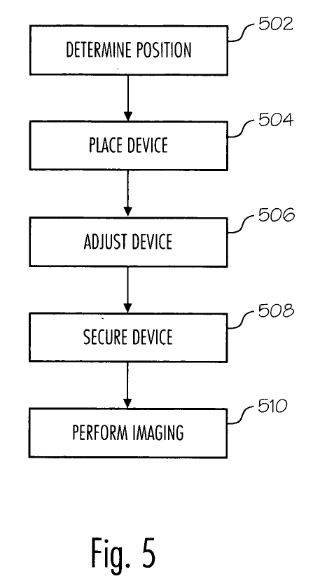

First and second bags 102 and 104 are connected by web section 106. Web section 106 can be any material that can connect first and second bag 102 and 104. In one exemplary embodiment, web 106 is manufactured using a stretchable or elastic material to achieve a better fit around a limb. In another exemplary embodiment, web 106 can be manufactured from the same or similar material used for cover portion 202. In one exemplary embodiment, web 106 is a solid piece of material. Web section 106, in another exemplary embodiment, comprises a series of straps or other non-solid pieces of material connecting first bag 102 and second bag 104. Web section 106 is designed to be large enough to fit underneath a limb, as will be discussed in further detail in conjunction with FIG. 3 and FIG. 4.

Straps 108 secure the device 100 around a limb. Straps 108 can be any devices that will secure the first bag 102 and second bag 104 on either side of a limb to assist in the positioning and immobilization of the limb. In one exemplary embodiments straps 108 are deployed in pairs with one of the pair of straps 108 affixed to first bag 102 and the second strap 108 of the pair affixed to second bag 104. Each of the pair of straps can then be connected, tied or otherwise fixed together to secure the device 100. In one exemplary embodiment, hook and loop fasteners, such as VELCRO, a fabric hook and loop fastener manufactured by Velcro of Manchester, N.H., can be utilized for ease in the securing of the straps 108. Other suitable connectors can also be used.

FIG. 3 illustrates the use of the device 100 to secure a limb 302. As seen in figure one, first bag 102 and second bag 104 are placed on either side of limb 302, assisting in the positioning and immobilization of the limb 302. The web section 106 is positioned underneath limb 302. The straps 108 affixed to the first bag 102 are connected to the straps from the second bag 104. In one exemplary embodiment, the straps are connected using a hook and loop fastener. Once the limb 302 is in position and secured, the diagnostic imagining can be performed.

In FIG. 3 an optional foot strap is illustrated. Foot strap 304 connects to one of the straps 108 of one of the bags and is placed around the foot and attached to a strap on the opposite bag. This holds the foot in a 90 degree flexion position for ankle and foot MRI-imaging.

FIG. 4 is a flowchart of an exemplary method for using device 10o. In first step 402, the proper positioning of the limb is determined. Next, in step 404, the web portion 106 of the device 100 is placed under the limb 302 and the first and second bags 102 and 104 are placed on the side of the limb. In step 406, the first and second bags 102 and 104 are adjusted to position and secure the limb 302. In step 408, the straps are used to secure the device 100. Then, in step 410, diagnostic imaging is completed.

While at least one exemplary embodiment has been presented in the foregoing detailed description of the invention, it should be appreciated that a vast number of variations exist. It should also be appreciated that the exemplary embodiment or exemplary embodiments are only examples, and are not intended to limit the scope, applicability, or configuration of the invention in any way. Rather, the foregoing detailed description will provide those skilled in the art with a convenient road map for implementing an exemplary embodiment of the invention, it being understood that various changes may be made in the function and arrangement of elements described in an exemplary embodiment without departing from the scope of the invention as set forth in the appended claims.

1. A device for positioning a limb for diagnostic imaging comprising:
a first bag filled with a medium;
a second bag filled with the medium; and
a web section connecting the first bag and the second bag.

2. The device of claim 1 wherein the medium is sand.

3. The device of claim 1 wherein the medium is dense gel.

4. The device of claim 1 wherein the web section is manufactured using an elastic material.

5. The device of claim 1 further comprising a plurality of straps, a portion of the plurality of straps connected to the first bag and the remainder of the plurality of straps connected to the second bag, the straps configured to secure the device to the limb.

6. The device of claim 5 wherein the straps include hook and loop fasteners for securing the portion and the remainder together.

7. The device of claim 1 wherein the first and second bag are manufactured from a santiziable material.

8. The device of claim 1 further comprising a foot strap for placing around a foot and holding the foot at a 90 degree flexion angle.

9. A device for positioning a limb for diagnostic imaging comprising:
a first bag configured to be placed against one side of the limb;
a second bag configured to be placed against the second side of a limb:
a web section connecting the first bag and the second bag, the web section placed underneath the limb; and
a plurality of straps, a portion of the plurality of straps connected to the first bag and the remainder of the plurality of straps connected to the second bag, the straps configured to secure the device to the limb.

10. The device of claim 9 wherein the first bag and second bag are filled with a medium.

11. The device of claim 10 wherein the medium is sand.

12. The device of claim 10 wherein the medium is dense gel.

13. The device of claim 9 wherein the web section is manufactured using an elastic material.

14. The device of claim 9 wherein the straps include hook and loop fasteners for securing the portion and the remainder together.

15. The device of claim 9 wherein the first and second bag are manufactured from a santiziable material.

16. The device of claim 9 further comprising a foot strap for placing around a foot and holding the foot at a 90 degree flexion angle.
